(19) United States
(12) Patent Application Publication
(10) Pub. No.: US 2025/0228701 A1
(43) Pub. Date: Jul. 17, 2025

(54) PESSARY WITH REMOVAL FEATURE

(71) Applicant: Tim Young, Moorabbin (AU)

(72) Inventor: Tim Young, Moorabbin (AU)

(73) Assignee: Sayco Pty Ltd, Moorabbin, Victoria (AU)

(21) Appl. No.: 19/170,237

(22) Filed: Apr. 4, 2025

Related U.S. Application Data
(63) Continuation of application No. PCT/AU2023/050989, filed on Oct. 6, 2023.

(30) Foreign Application Priority Data
Oct. 6, 2022 (AU) 2022902906

(51) Int. Cl. A61F 6/08

(52) U.S. Cl. CPC A61F 6/08

(57) ABSTRACT

A pessary device includes a body and a finger-pull disposed on and extending from the body and is adapted to receive at least one finger of a user to facilitate removal of the pessary device. The finger-pull includes a loop extending from an outwardly facing surface of the body, in use, and an opposed anchoring section connected or integral with the outwardly facing surface. A method of producing a pessary device includes providing a finger-pull including a loop and an opposing anchoring section. The method includes forming a pessary body around the anchoring section, thereby securing the finger-pull to the pessary body.

### CROSS-REFERENCE TO RELATED PATENT APPLICATIONS

This patent application is a continuation of PCT/AU2023/050989, filed Oct. 6, 2023, which claims priority to Australian Application No. 2022902906, filed Oct. 6, 2022, the entire teachings and disclosure of both applications are incorporated herein by reference thereto.

### FIELD OF INVENTION

The present invention relates to a pessary with a removal feature that facilitates removal of the pessary from a user. More particularly, the invention relates to a pessary that includes a finger-pull.

### BACKGROUND ART

Pessaries are implantable devices that have been used for many decades for the treatment of Pelvic Organ Prolapse and Stress Urinary Incontinence. Pessaries are mainly categorized into two types, supporting pessaries and space-occupying pessaries. Support pessaries function by supporting the prolapse and space-occupying pessaries by filling the vaginal space. There are also lever type pessaries. For example, pessaries may include at least the following types: ring, ring with support, shelf, Shaatz, Gellhorn, Risser, Smith, cube, tandem cube, cup, oval, Hodge, Hodge with knob, Hodge with support, Gehrung, incontinence dish with support, donut, incontinence ring, incontinence dish, and Inflatoball and Inflatable Donut.

The most common types of pessaries are the ring pessary, available with and without support, and the Gellhorn pessary, available with short and long stems. Notably, ring pessaries are normally used for minor prolapses and Gellhorn pessaries are used for moderate to severe prolapses.

The ring pessary in its simplest form is a circular ring of inert material, such as PVC or latex, or silicone. However, ring pessaries often include a supporting membrane that extends between the inner edges of the ring. The supporting membrane generally includes a web of material extending within the boundary of the ring and having drain holes in the supporting membrane. Ring pessaries can generally be easily folded in half for insertion and may be removed by a user by hooking a finger under or over the rim of the ring pessary, tilting the ring pessary slightly, and gently pulling down and out of the vagina. However, this is not possible with a ring with a supporting membrane.

The Gellhorn pessary has a circular base normally made out of silicone, about 3-5 mm thick. The base is slightly concave as this form assists in creating suction and keeping the Gellhorn in place. Similar to the ring pessary with support, the Gellhorn has drain holes in the base. The Gellhorn pessary is distinctive in that it includes a central stem located on the base of the Gellhorn pessary, the stem including a spherical knob at the distal end thereof.

The Gellhorn pessary is a widely used pessary. To insert the Gellhorn pessary the stem, which is flexible, is bent or compressed to the base and the base is inserted in-line with the vaginal opening. Removal of the Gellhorn pessary is generally difficult due to the suction created by the concave circular base of the pessary. Clinicians often need to use a device such as sponge forceps to grip the stem, placing a finger up and around the base to release the suction, and move the base downwards to remove the pessary. As such, in its current form the Gellhorn pessary is difficult to remove by a clinician and is generally considered to be a pessary that a patient cannot remove by themselves.

Another widely used space-occupying pessary is the cube pessary. This type of pessary includes a cube body, generally including drain holes, and a cord for grasping in order to remove the pessary. The cord is generally attached to the cube body with an anchor. The cord may also be looped with two anchors securing each end of the loop to the cube body through the drain holes. However, such cord and anchor systems have the disadvantage of potential breakage to leave material inside the user after removal of the cube pessary and making the pessary difficult to remove for the user. In addition, pulling on the cord without deforming the walls of the cube pessary (thereby breaking the suction) may cause the pessary to pull the prolapse down as it is removed.

Generally, these types of pessaries have been used in their current forms for a number of decades. More particularly, space-filling pessaries such as the cube and Gellhorn pessaries have been used in their present form with little consideration given to facilitating ease of removal by a user.

The subject matter claimed herein is not limited to embodiments that solve any disadvantages or that operate only in environments such as those described above. Rather, this background is only provided to illustrate exemplary technology areas where some embodiments described herein may be practiced.

Various aspects and embodiments of the invention will now be described.

### SUMMARY OF INVENTION

As mentioned above, the present invention relates generally to a pessary with a removal feature that facilitates removal of the pessary from a user. More particularly, the invention relates to a pessary that includes a finger-pull.

According to one aspect of the invention there is provided a pessary device comprising:
a body;
a finger-pull disposed on and extending from the body portion and adapted to receive at least one finger of a user to facilitate removal of the pessary device
wherein the finger-pull comprises a loop extending from an outwardly facing surface of the body, in use, and an opposed anchoring section connected or integral with the outwardly facing surface.

The body of the pessary device is not particularly limited and may take any suitable form. For example, the body may comprise a pessary body selected from a ring, ring with support, shelf, Shaatz, Gellhorn, Risser, Smith, cube, tandem cube, cup, oval, Hodge, Hodge with knob, Hodge with support, Gehrung, dish, dish with support and donut.

In one embodiment, the anchoring section preferably includes two ends connected or integral with the outwardly facing surface, the distance between the two ends of the loop being less than a diameter of the loop, preferably wherein the distance between the two ends is about half the diameter of the loop. The contact point of the ring pull to the pessary needs to be relatively narrow. This ensures the force applied on the body of the pessary device is generally focused at a region or point on the pessary device. This allows a user to apply less force to cause deformation of the pessary device for easy removal. The anchoring section is more preferably disposed at or towards an edge of the outwardly facing surface. This further minimises the force required to break suction between the body of the pessary device and the vaginal wall.

The anchoring section of the pessary device may further comprise a supporting web extending between the two ends, the supporting web preferably comprising a substantially triangular or V-shaped web extending between the two ends. This supporting web advantageously minimises deformation of the finger-pull and transfers the force applied to the body of the pessary device.

The contact point between the loop and the body of the pessary device may be thickened and/or reinforced to prevent and avoid breakages of the material of the loop. Furthermore, the thickness of the loop may be predetermined to minimise the stretching of the loop, thereby minimising the force required to remove the pessary device.

Preferably, the loop is disposed at an angle towards the centre of the pessary device. This advantageously ensures the loop does not cause patient discomfort. The angle may vary based on the pessary type and size and the moulding form.

In preferred embodiments, the anchoring section is connected to the outwardly facing surface of the body of the pessary device by bonding to the outwardly facing surface or anchoring beneath the outwardly facing surface. In a particular embodiment, the anchoring section of the finger-pull further comprises an anchor extending from or disposed on the anchoring section and anchored beneath the outwardly facing surface of the body of the pessary device. The anchor may comprise, for example, an enlarged bulbous section, t-shaped section or disc disposed. It may also comprise barbs disposed on the anchoring section.

In order to reinforce the strength of the finger-pull, and particularly the loop, the finger-pull is preferably formed from a material having a harder Shore rating than the body of the pessary device. The Shore hardness of the body is preferably from 25-35, more preferably about 30, while the Shore hardness of the finger-pull (particularly the loop) is preferably about 60-70, more preferably about 70.

To facilitate identification of the loop for removal of the pessary device, the loop is preferably formed of a material of a different colour to the body of the pessary device.

It is envisaged that the loop or ‘ring pull’ type finger-pull will be particularly suitable for space-occupying pessaries. As such, the body preferably comprises a pessary body selected from a Shaatz, Gellhorn, cube, tandem cube or donut, and the application of user pulling force on the loop deforms the body thereby releasing suction between outer edges of the pessary body and vaginal cavity surfaces they are contacting, in use.

As is conventional, the body and finger-pull are preferably formed from silicone. In certain embodiments the finger-pull in its entirety is formed with the pessary body as part of the pessary moulding process. That is, the pessary device may be a unitary piece of material, generally silicone material. However, as discussed above, the anchoring section is preferably formed from a harder material than the body of the pessary device and is anchored to the body of the pessary device.

According to another aspect of the invention there is provided a method of producing a pessary device comprising:
providing a finger-pull comprising a loop and an opposing anchoring section; and
forming a pessary body around said anchoring section, thereby securing said finger-pull to said pessary body.

The forming of the pessary body preferably comprises moulding the pessary body around the anchoring section. Preferably, the anchoring section is disposed and held at a predetermined angle relative to the pessary body during moulding.

As with the previous aspect of the invention, the pessary body may selected from a ring, ring with support, shelf, Shaatz, Gellhorn, Risser, Smith, cube, tandem cube, cup, oval, Hodge, Hodge with knob, Hodge with support, Gehrung, dish, dish with support and donut.

The anchoring section preferably includes two ends of the loop, the distance between the two ends of the loop being less than a diameter of the loop, preferably wherein the distance between the two ends is about half the diameter of the loop. The anchoring section may further comprise a substantially V-shaped supporting web extending between the two ends, the supporting web preferably comprising a substantially triangular web extending between the two ends.

The anchoring section is preferably disposed at or towards an edge of the outwardly facing surface. The loop is preferably disposed at an angle towards the centre of the pessary device.

The anchoring section of the finger-pull comprises an anchor. The anchor may comprise an enlarged bulbous section, t-shaped section or disc, or may comprise barbs located on the anchoring section.

The finger-pull is preferably formed from a material having a harder Shore rating than the body of the pessary device. Preferably, the Shore hardness of the body is from about 25-35, more preferably about 30, and the Shore hardness of the finger-pull is about 60-70, more preferably about 70.

The loop is preferably formed of a material of a different colour to the body of the pessary device. The body and finger-pull are preferably formed from silicone.

### BRIEF DESCRIPTION OF ACCOMPANYING DRAWINGS

To further clarify various aspects of some embodiments of the present invention, a more particular description of the invention will be rendered by references to specific embodiments thereof, which are illustrated in the appended drawings. It should be appreciated that these drawings depict only typical embodiments of the invention and are therefore not to be considered limiting on its scope. The invention will be described and explained with additional specificity and detail through the accompanying drawings in which:

FIG. 1 illustrates a perspective view of a pessary device accordingly to a first embodiment of the invention, including a Gellhorn pessary body.

FIG. 2 illustrates a rear view of the pessary device of FIG. 1 showing the finger-pull in closer detail.

FIG. 3 illustrates a perspective view of an alternative pessary device to that illustrated in FIGS. 1 and 2, including a cube pessary body instead of a Gellhorn pessary body.

### DETAILED DESCRIPTION OF THE PREFERRED EMBODIMENTS

Hereinafter, this specification will describe the present invention according to the preferred embodiments. It is to be understood that limiting the description to the preferred embodiments of the invention is merely to facilitate discussion of the present invention and it is envisioned without departing from the scope of the appended claims.

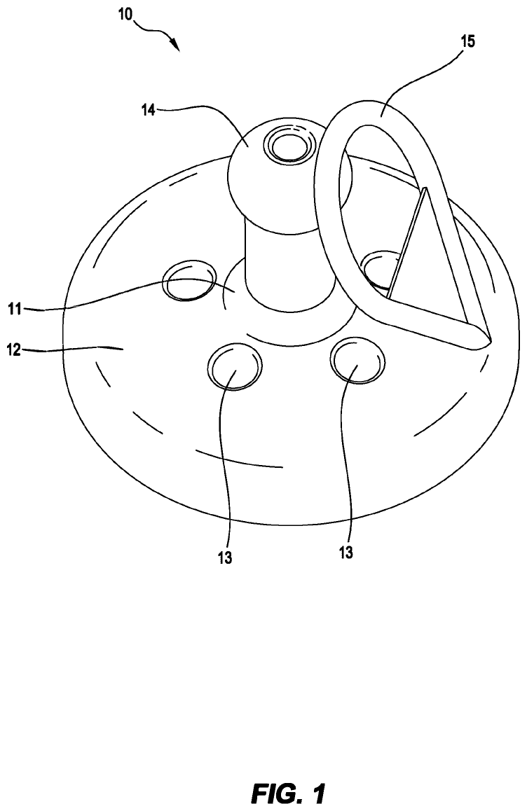

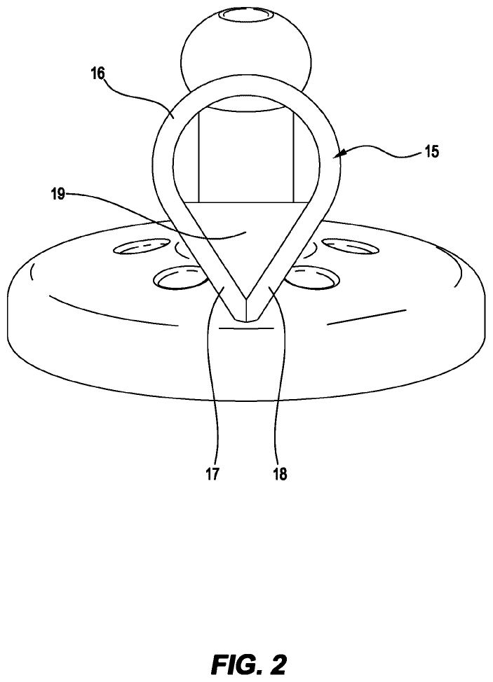

Referring to FIGS. 1 and 2, a pessary device 10 is illustrated. The pessary device 10 includes a Gellhorn pessary body 11 that includes a base 12 provided with a plurality of drain holes 13 and a stem 14 extending therefrom. The Gellhorn pessary body 11 may vary in dimensions as required for the end user. The base 12 is adapted to contact the vaginal cavity walls establishing a seal with the vaginal cavity walls. As previously noted, this makes it generally impractical for a user of a conventional Gellhorn pessary device to remove it themself.

To address the difficulties associated with removal of a conventional Gellhorn pessary, the pessary device 10 includes a finger-pull 15 on a surface thereof. The surface is one that faces outwards from the vaginal cavity in use. The finger-pull 15 includes a loop 16, and an anchoring section including ends 17,18 that are attached or integral with the base 12. The distance between the ends 17,18 of the loop 16 is relatively small. This ensures that the force applied by a user on pulling the loop 16 is focused on a specific location on the base 12. Also, the ends 17,18 of the loop 16 are located at or towards an outer edge of the base 12. This further ensures that the force applied by the user is focused on a specific outer location of the base 12. The distance between the ends 17,18 of the loop 16 and the positioning of the ends 17,18 at or towards an outer edge of the base 12 individually or in combination aid in the deformation of the base 12 during removal to facilitate breaking of the seal between the outer edges of the base 12 and the vaginal cavity wall.

As best illustrated in FIG. 1, the loop 16 is disposed at an angle to the base 12. Specifically, the loop 16 is disposed at a downward angle towards the centre of the base 12. The angle of the loop 16 acts in two ways. Firstly, a user may place a finger in the loop 16 and also place pressure on the stem 14 of the Gellhorn pessary body 11, thereby increasing deformation of the base 12 aiding in the removal of the pessary device 10. Also, as the loop 16 is angled downward, it does not interfere with internal walls of the vaginal cavity ensuring more comfort while the pessary device 10 is in use.

The anchoring section of the finger-pull 15 is provided with a substantially V-shaped supporting web 19 that extends between the ends 17,18 and is anchored to or integral with the base 12 between the ends 17,18. This provides support to the loop 16 and minimises deformation of the loop 16 on application of force by the user on removal of the pessary device 10. Again, this advantageously ensures that maximum deformation of the base 12 is achieved making it easier to dislodge the edges of the base 12 from the vaginal cavity walls. This also reduces or eliminates the chances of breakage of the ends 17,18 of the loop 16.

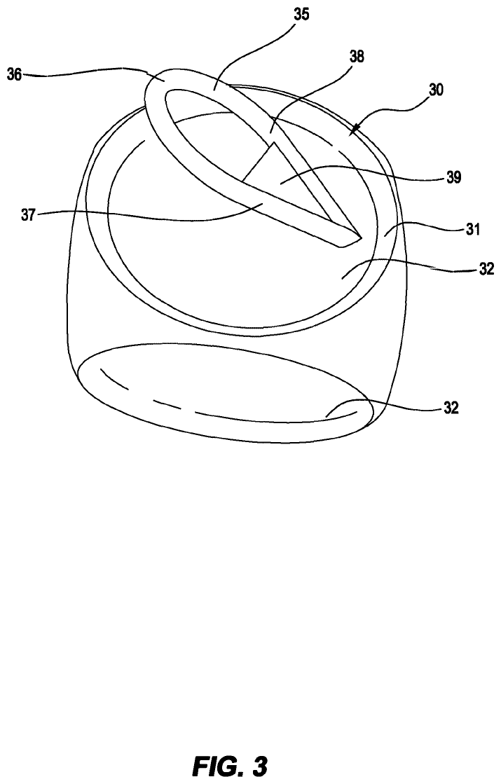

Referring to FIG. 3, an alternative pessary device 30 is illustrated. This pessary device 30 includes a cube pessary body 31 in place of the Gellhorn pessary body 11 previously discussed in respect of FIGS. 1 and 2. The cube pessary body 11 acts in a similar fashion to that of the Gellhorn pessary body 11, in that is acts as a space-filling device in the vaginal cavity. The cube pessary body 31 includes faces 32, each of which includes a concave surface. This is consistent with conventional cube pessaries.

As was the case with the pessary device 10 of FIGS. 1 and 2, the pessary device 30 includes a finger-pull 35 attached or integral with a face of the cube pessary body 31 that is intended to face outwardly from the vaginal cavity in use. The finger-pull 35 is of the same form as that previously described and includes a loop 36 and an anchoring section including ends 37,38 of the loop 36 and a substantially V-shaped supporting web 39 disposed therebetween. Once again, the ends 37,38 of the loop 36 are disposed at or towards and edge of the outward face of the cube pessary body 31. The reasons are consistent with those outlined above, including focusing force applied by the user and minimising deformation of the loop 36 on the application of this force. Again, the aim is to facilitate deformation of the cube pessary body 31 to dislodge this from the vaginal cavity walls.

While it is considered the above finger-pull 15,35 may be applied to any form of pessary body, including but not limited to Shaatz, Gellhorn, Risser, Smith, cube, tandem cube, cup, oval, Hodge, Hodge with knob, Hodge with support, Gehrung, dish, dish with support and donut pessary bodies, it is considered that this form of finger-pull 15,35 may be best suited to space-filling pessaries. As such, while only Gellhorn and cube pessaries are illustrated, it is envisaged this form of finger-pull 15,35 will be equally applicable to at least Shaatz, Gellhorn, shelf, cube, tandem cube and donut pessary bodies.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

Unless the context requires otherwise or specifically stated to the contrary, integers, steps or elements of the invention recited herein as singular integers, steps or elements clearly encompass both singular and plural forms of the recited integers, steps or elements.

It will be appreciated that the foregoing description has been given by way of illustrative example of the invention and that all such modifications and variations thereto as would be apparent to persons of skill in the art are deemed to fall within the broad scope and ambit of the invention as herein set forth.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pessary device comprising:
a body; and
a finger-pull disposed on and extending from the body and adapted to receive at least one finger of a user to facilitate removal of the pessary device, said finger-pull comprising a loop extending from an outwardly facing surface of the body, in use, and an opposed anchoring section connected or integral with the outwardly facing surface,
wherein the anchoring section is disposed at or towards an edge of the outwardly facing surface aiding in the deformation of the base during removal of the pessary device to facilitate breaking of a seal between the body of the pessary device and a vaginal cavity wall.

2. The pessary device of claim 1, wherein the body comprises a pessary body selected from a ring, ring with support, shelf, Shaatz, Gellhorn, Risser, Smith, cube, tandem cube, cup, oval, Hodge, Hodge with knob, Hodge with support, Gehrung, dish, dish with support and donut.

3. The pessary device of claim 1, wherein the anchoring section includes two ends of the loop connected or integral with the edge of the outwardly facing surface, the distance between the two ends of the loop being less than a diameter of the loop, preferably wherein the distance between the two ends is about half the diameter of the loop.

4. The pessary device of claim 3, wherein the anchoring section further comprises a substantially V-shaped supporting web extending between the two ends, the supporting web preferably comprising a substantially triangular web extending between the two ends.

5. The pessary device of claim 1, wherein the loop is disposed at an angle towards the centre of the pessary device from the edge of the outwardly facing surface.

6. The pessary device of claim 1, wherein said anchoring section is connected to the outwardly facing surface of the body of the pessary device by bonding to the edge of the outwardly facing surface or anchoring beneath the outwardly facing surface.

7. The pessary device of claim 6, wherein said anchoring section of said finger-pull further comprises an anchor that is disposed beneath the outwardly facing surface of the body of the pessary device.

8. The pessary device of claim 7, wherein the anchor comprises an enlarged bulbous section, t-shaped section or disc, or comprises barbs located on said anchoring section.

9. The pessary device of claim 1, wherein said finger-pull is formed from a material having a harder Shore rating than the body of the pessary device, preferably wherein the Shore hardness of the body is from about 25-35, more preferably about 30, and the Shore hardness of the finger-pull is about 60-70, more preferably about 70.

10. The pessary device of claim 1, wherein said loop is formed of a material of a different colour to said body of the pessary device.

11. The pessary device of claim 1, wherein the body comprises a pessary body selected from a Shaatz, Gellhorn, cube, tandem cube and donut.

12. The pessary device of claim 1, wherein the body of the pessary device is a unitary piece of material, preferably formed from silicone.

13. The pessary device of claim 1, wherein the body and finger-pull are formed from silicone.

14. A method of producing a pessary device comprising:
providing a finger-pull comprising a loop and an opposing anchoring section; and
forming a pessary body around said anchoring section, thereby securing said finger-pull to an outwardly facing surface of said pessary body,
wherein the anchoring section is disposed at or towards an edge of the outwardly facing surface.

15. The method of claim 14, wherein said forming of the pessary body comprises moulding said pessary body around the anchoring section.

16. The method of claim 15, wherein said anchoring section is disposed and held at a predetermined angle relative to the pessary body during moulding.

17. The method of claim 14, wherein the pessary body comprises a pessary body selected from a ring, ring with support, shelf, Shaatz, Gellhorn, Risser, Smith, cube, tandem cube, cup, oval, Hodge, Hodge with knob, Hodge with support, Gehrung, dish, dish with support and donut.

18. The method of claim 14, wherein the anchoring section includes two ends of the loop, the distance between the two ends of the loop being less than a diameter of the loop, preferably wherein the distance between the two ends is about half the diameter of the loop.

19. The method of claim 18, wherein the anchoring section further comprises a substantially V-shaped supporting web extending between the two ends, the supporting web preferably comprising a substantially triangular web extending between the two ends.

20. The method of claim 14, wherein the anchoring section is disposed at or towards an edge of the outwardly facing surface.

21. The method of claim 14, wherein the loop is disposed at an angle towards the centre of the pessary device.

22. The method of claim 14, wherein said anchoring section of said finger-pull comprises an anchor.

23. The method of claim 22, wherein the anchor comprises an enlarged bulbous section, t-shaped section or disc, or comprises barbs located on said anchoring section.

24. The method of claim 14, wherein said finger-pull is formed from a material having a harder Shore rating than the body of the pessary device, preferably wherein the Shore hardness of the body is from about 25-35, more preferably about 30, and the Shore hardness of the finger-pull is about 60-70, more preferably about 70.

25. The method of claim 14, wherein said loop is formed of a material of a different colour to said body of the pessary device.

26. The method of claim 14 wherein the body and finger-pull are formed from silicone.
